(19) United States
(12) Patent Application Publication
(10) Pub. No.: US 2010/0035270 A1
(43) Pub. Date: Feb. 11, 2010

(54) Methods and Compositions for the Detection of Bovine Pregnancy

(76) Inventors: Thomas R. Hansen, Fort Collins, CO (US); Kathleen J. Austin, Laramie, WY (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/536,189

(22) Filed: Aug. 5, 2009

Related U.S. Application Data
(63) Continuation of application No. 11/542,056, filed on Oct. 2, 2006, now abandoned.
(60) Provisional application No. 60/722,530, filed on Sep. 30, 2005.

(51) Int. Cl. C12Q 1/68; G01N 33/566; C40B 40/08; C07K 16/18

(52) U.S. Cl. 435/6; 436/501; 506/17; 530/387.1

(57) ABSTRACT

Provided herein are pregnancy specific marker genes, such as those shown in Tables I-III, and methods of detecting the same to determine bovine pregnancy.

This application claims priority under 35 U.S.C. §119(e) to U.S. Provisional Application No. 60/722,530, filed on Sep. 30, 2005. The entire contents of the '530 application are incorporated herein by reference as though set forth in full.

### FIELD OF THE INVENTION

This invention relates to the field of molecular biology and reproductive biology. More specifically, the present invention provides materials and methods for rapid and efficient detection of bovine pregnancy.

### BACKGROUND OF THE INVENTION

Several publications and patent documents are cited throughout the specification in order to describe the state of the art to which this invention pertains. Each of these citations are incorporated herein by reference as though set forth in full.

Reproductive efficiency (time from calving to conception), feed costs associated with maintaining non-pregnant cows, annual milk production (dairy) and weaning weights (beef) are major constraints for optimization of management in bovine industries. Accordingly, early and accurate detection of pregnancy are critical to efficient cattle management. However, currently there is no rapid and reliable bovine pregnancy test.

Human chorionic gonadotropin (Fishel S B, et al. (1984) Science 223:816-8) is present in high amounts, in the urine of pregnant humans, and is the basis for the rapid pregnancy test that is sold commercially. No corresponding chorionic gonadotropin protein has been identified in bovine blood or urine.

There are many methods of determining pregnancy in cows, but they all have some difficulty associated with them. Mechanical methods, which detect actual fetuses, are reasonably accurate, but cannot be conducted early in pregnancy. There are two such methods commonly used. The first is through rectal palpation for presence of the fetus (Sasser R G, et al. (1987) J Reprod Fertil Suppl 34:261-71; Beal W E, et al. (1992) J Anim Sci 70:924-9; Fricke P M (2002) J Dairy Sci 85:1918-26; Hanzen C, et al. (1987) Vet Rec 121:200-2). This method is accurate after 40-50 days of pregnancy. In some cases skilled technicians can determine pregnancy using this method as early as 35 days, but this is not recommended, because manipulation of the uterus and the fetal membranes may cause abortion. The second common method is ultrasound. Ultrasound is accurate as early as day 27 of pregnancy, but also requires a skilled technician (Sasser R G, et al. (1987) J Reprod Fertil Suppl 34:261-71; Beal W E, et al. (1992) J Anim Sci 70:924-9; Fricke P M (2002) J Dairy Sci 85:1918-26; Hanzen C, et al. (1987) Vet Rec 121:200-2). Both of these methods must be performed relatively late following establishment of pregnancy, which occurs between days 14 to 19 (Thatcher W W, et al. (1995) J Reprod Fertil Suppl 49:15-28; Bazer F W, et al. (1991) J Reprod Fertil Suppl 43:39-47; Helmer S D, et al. (1989) J Reprod Fertil 87:89-101; Bazer F W, et al. (1986) J Reprod Fertil 76:841-50; Thatcher W W, et al. (1986) J Anim Sci 62 Suppl 2:25-46).

Other means of pregnancy testing involve measuring hormone or chemical changes that occur during pregnancy. Early methods were based on detecting the steroid hormone, progesterone. While these techniques still have merit and utility (Booth J M, et al. (1979) Br Vet J 135:478-88; Holdsworth R J, (1979) Br Vet J 135:470-7; Pengelly J (1979) Vet Rec 104:328; van de Wiel D F, et al. (1978) Tijdschr Diergeneeskd 103:91-103; Macfarlane J S, et al. (1977) Vet Rec 100:565-6; Dobson H, et al. (1976) Br Vet J 132:538-42; Hoffmann B, et al. (1976) Br Vet J 132:469-76), timing of progesterone tests is difficult, and improper timing can lead to an incorrect determination of pregnancy status.

The luteal phase of the estrous cycle is the time between ovulation and luteolysis (characterized by the disintegration of the corpus lutum). Progesterone is released into the milk, and also circulates in the blood during the luteal phase of the estrous cycle, and during pregnancy. In a well timed blood test, a low concentration of progesterone is interpreted to reflect a non-pregnant cow that is undergoing luteolysis in preparation for the next estrous cycle. However, it is difficult to distinguish a pregnant cow from a non-pregnant cow in the luteal phase of estrous. This is because if a non-pregnant cow is still in the luteal phase when she is tested, her progesterone levels will be similar to those of a pregnant cow. Missing luteolysis by one or two days contributes to a high rate of false positive tests (a cow determined to be pregnant by the test, but actually being non-pregnant).

One technique to improve the accuracy of progesterone tests involves determining progesterone concentration on the day of artificial insemination and then again three weeks later on day 21. Because most estrous cycles are 16 to 24 days in length with an average of 21 days, then it is possible to sample most non-pregnant cows at a time that progesterone concentration would be low. This helps distinguish the pregnant cows from non-pregnant cows, but still does not provide accurate and reliable results (Sasser R G, et al. (1987) J Reprod Fertil Suppl 34:261-71; Pitcher P M, et al. (1990) J Am Vet Med Assoc 197:1586-90; Oltenacu P A, et al. (1990) J Dairy Sci 73:2826-31; Nebel R L (1988) J Dairy Sci 71:1682-90; Gowan E W, et al. (1982) J Dairy Sci 65:1294-1302).

Another pregnancy specific marker, Early pregnancy factor (EPF) (Ito K, et al. (1998) Am J Reprod Immunol 39:356-61; Cavanagh A C, et al. (1994) Eur J Biochem 222:551-60; Sakonju I, et al. (1993) J Vet Med Sci 55:271-4; Klima F, et al. (1992) J Reprod Immunol 21:57-70) also has been called Early Conception Factor (ECF) (Gandy B, et al. (2001) Theriogenology 56:637-47; Cordoba M C, et al. (2001) J Dairy Sci 84:1884-9; Nancarrow C D, et al. (1981) J Reprod Fertil Suppl 30:191-9) was first described by its ability to inhibit rosette formation between T lymphocytes and red blood cells. This bioassay was used to detect pregnancy in ruminants, but never was developed fully into a useful diagnostic test, because the specific protein that had this unique activity was difficult to purify. More recently, EPF has been shown to be a member of the chaperonin 10 gene family (Cavanagh A C, et al. (1994) Eur J Biochem 222:551-60). However, two recent studies by independent laboratories have shown that EPF is not a very useful diagnostic for early pregnancy (Gandy B, et al. (2001) Theriogenology 56:637-47; Cordoba M C, et al. (2001) J Dairy Sci 84:1884-9).

Yet another putative pregnancy marker, Pregnancy-Specific Protein B, is reported to be present in binucleate cells of the trophoblast as early as day 21 of pregnancy in cows (Sasser R G, et al. (1986) Biol Reprod 35:936-42; Humblot F, et al. (1988) J Reprod Fertil 83:215-23; Sasser R G, (1989) J Reprod Fertil Suppl 37:109-13; Kiracofe G H, et al. (1993) J Anim Sci 71:2199-205; Szenci O, et al. (1998) Theriogenology 50:77-88). The PSPB is a member of the Pregnancy Associated Glycoprotein or PAG family (Zoli A P, et al. (1992) Biol Reprod 46:83-92; Xie S, et al. (1994) Biol Reprod 51:1145-53; Roberts R M, et al. (1995) Adv Exp Med Biol 362:231-40; Green J A, et al. (2000) Biol Reprod 62:1624-31; Perenyi Z S, et al. (2002) Reprod Domest Anim 37:100-4; Sousa N M, et al. (2002) Reprod Nutr Dev 42:227-41; de Sousa N M, et al. (2003) Theriogenology 59:1131-42; Karen A, et al. (2003) Theriogenology 59:1941-8). Specifically, it is identical to PAG-1 (Xie S, et al. (1994) Biol Reprod 51:1145-53; Roberts R M, et al. (1995) Adv Exp Med Biol 362:231-40). There are now 20 different PAG genes that have been identified (Roberts R M, et al. (1995) Adv Exp Med Biol 362:231-40). However, detection of this protein in blood is not accurate until after day 30 and this protein has a very long half-life, so it remains in circulation for several months following parturition, which limits its utility in post-partum cows. When cows are mated or inseminated prior to 70 days post partum, residual post-partum PSPB concentrations (from previous pregnancy) lowers the accuracy of using PSPB as a marker for pregnancy (Kiracofe G H, et al. (1993) J Anim Sci 71:2199-205; Sasser R G, et al. (1988) J Anim Sci 66:3033-9).

Two other blood cell markers have been proposed to be useful for determining pregnancy status in cows (See US Patent US Application No: 2003/10224452 by Colgin et al.). In this disclosure, two interferon- and pregnancy-induced genes called ISG15 and MX2 have been described as useful markers for pregnant cows. Also, lower levels of ISG15 and MX2 expression in blood indicate cows that are not pregnant. These interferon-induced genes and gene products are not the only markers for pregnancy status in blood from ruminants.

In light of the criticality of early and accurate pregnancy determination in efficient cattle production, and the current lack of an early and accurate means of determining pregnancy in cattle, a need exists for the further characterization of genes which are differentially expressed in pregnant animals.

### SUMMARY OF THE INVENTION

In accordance with the present invention, methods and compositions for detecting bovine pregnancy are provided. Specifically, pregnancy specific markers are provided, as well as methods of determining bovine pregnancy by detecting differential expression of the same.

One embodiment of the invention comprises at least one isolated, enriched, or purified nucleic acid molecule which is differentially expressed in pregnant bovines, or which encodes a pregnancy specific marker, said at least one nucleic acid molecule preferably being affixed to a solid support. A nucleic acid molecule encoding a pregnancy specific marker includes any nucleic acid molecule which encodes any protein which is a variant or derivative of a pregnancy specific marker, and which retains pregnancy specific marker function. Exemplary pregnancy specific marker nucleic acid molecules are listed in Tables I-III.

Also provided in accordance with the invention are oligonucleotides, including probes and primers, that specifically hybridize with the nucleic acid sequences set forth above.

In a further aspect of the invention, recombinant DNA molecules comprising the nucleic acid molecules set forth above, operably linked to a vector are provided. The invention also encompasses host cells comprising a vector encoding a pregnancy specific marker of the invention.

In another aspect of the invention, methods for detecting pregnancy specific marker molecules in a biological sample are provided. Such molecules can be pregnancy specific marker nucleic acids, such as mRNA, DNA, cDNA, or pregnancy specific marker polypeptides or fragments thereof. Exemplary methods comprise detection of isolated biological molecules which hybridize to pregnancy specific markers which are affixed to a solid support, or mRNA analysis, for example by RT-PCR. Immunological methods include for example contacting a sample with a detectably labeled antibody immunologically specific for a pregnancy specific marker polypeptide and determining the presence of the polypeptide as a function of the amount of detectably labeled antibody bound by the sample relative to control cells. In a preferred embodiment, these assays may be used to detect a sequence as set forth in Tables I-III, or a protein encoded by the same.

In a further aspect of the invention, kits for detection of bovine pregnancy are provided. An exemplary kit comprises a pregnancy specific marker protein, polynucleotide or a gene chip comprising a plurality of such polynucleotides, or antibody, which are optionally linked to a detectable label. The kits may also include a pharmaceutically acceptable carrier and/or excipient, a suitable container, and instructions for use.

### BRIEF DESCRIPTION OF THE DRAWINGS

FIG. 1. FIG. 1A: Preferred bovine pregnancy marker molecules from blood cells. Columns represent fold change in pregnant cows when compared to non-pregnant cows, the P Value, an abbreviated name and the NCBI Accession number. FIGS. 1B-1F: Real Time PCR confirmation of upregulated genes from FIG. 1A in the blood cells from day 18 pregnant cows when compared to non-pregnant cows. Three day 18 pregnant and three day 18 non-pregnant cows, different from those used in FIG. 1A, are represented using specific oligonucleotide primers for each target.

### DETAILED DESCRIPTION OF THE INVENTION

In accordance with the present invention, compositions and methods are provided for the detection of pregnancy in ruminant animals, preferably bovines. A series of nucleic acid sequences which exhibit differential expression in response to pregnancy, and proteins encoded thereby, are used to advantage in a variety of assays as pregnancy specific markers for the rapid and efficient differentiation between pregnant and non-pregnant animals. These sequences are provided in Tables I-III. Markers within these tables that have P values less than P<0.01 are preferred markers.

Suitable assays for pregnancy detection include, without limitation, detection of the polynucleotides disclosed herein which are immobilized on Affymetrix Bovine Gene Chips, PCR, nucleic acid hybridization assays (e.g., Northern and Southern blotting), immunoassays, and Western Blotting.

### I. Definitions

The following definitions are provided to facilitate an understanding of the present invention:

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“Pregnancy specific marker” is a marker which is differentially expressed in pregnant animals versus non-pregnant animals. “Bovine pregnancy specific marker molecule” is a marker which is differentially expressed in pregnant bovines, compared to non-pregnant bovines. “Bovine pregnancy inducible marker molecule” is a marker which is induced, or caused to be expressed directly or indirectly in response to pregnancy. Such markers may include but are not limited to nucleic acids, proteins, or other small molecules.

The term “surrogate marker” of pregnancy is a marker which is directly or indirectly differentially expressed in response to pregnancy. Specifically, a surrogate marker may be any gene expression product which is differentially expressed in pregnant animals. A surrogate marker can be a polynucleotide, a protein, or any gene expression product, but is preferably an mRNA or protein expression product. Preferably, a surrogate marker of pregnancy is one which is differentially expressed in early pregnancy, for example on days 15-22 of pregnancy, with a preferred testing date prior to day 21 of pregnancy.

The term “early pregnancy” refers to a stage of pregnancy where a trophoblast has developed, but the fetuses are not yet detectable by mechanical means, such as ultrasound, radiograph, palpation. Optionally, “early pregnancy” may refer to any time before 4 weeks.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

The term “nucleic acid molecule” describes a polymer of deoxyribonucleotides (DNA) or ribonucleotides (RNA). The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. By the use of the term “enriched” in reference to nucleic acid it is meant that the specific DNA or RNA sequence constitutes a significantly higher fraction (2-5 fold) of the total DNA or RNA present in the cells or solution of interest than in normal or non-pregnant bovine cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any pregnancy specific marker gene, but does not hybridize to other bovine nucleotides. Also polynucleotide which “specifically hybridizes” may hybridize only to a pregnancy specific marker, such a pregnancy specific marker shown in Tables I-III. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the pregnancy specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the pregnancy specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, periplasm or peroxisome localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long. “Sample” or “patient sample” or “biological sample” generally refers to a sample which may be tested for a particular molecule, preferably a pregnancy specific marker molecule, such as a marker shown in Tables I-III. Samples may include but are not limited to cells, including uterine cells, uterine tissue, cervical tissue, chorionic villi, and body fluids, including blood, serum, plasma, urine, saliva, tears, pleural fluid and the like.

### II. Pregnancy Specific Marker Nucleic Acid Molecules, Probes, and Primers and Methods of Preparing the Same

Encompassed by the invention are isolated, enriched, or purified pregnancy specific marker nucleic acid molecules including, fragments, derivatives, mutants, and modifications of the same. Preferably, the pregnancy specific marker nucleotide is a marker shown in Table I-III. More preferably, the pregnancy specific nucleotide marker is affixed to a Gene Chip.

Pregnancy specific marker polynucleotides can be any one of, or any combination of the markers shown in Tables I-III, and further may include variants which are at least about 75%, or 80% or 85% or 90% or 95%, and often, more than 90%, or more than 95% homologous to the markers shown in Table I, over the full length sequence. Pregnancy specific marker polynucleotides also may be 60% or 65% or 70% or 75% or 80% or 85% or 90% or 95% or 97% or 98% or 99% or greater than 99% homologous to the markers shown in Tables I-III, over the full length sequence. All homology may be computed by algorithms known in the art, such as BLAST, described in Altschul et al. (1990), J. Mol. Biol. 215:403-10, or the Smith-Waterman homology search algorithm as implemented in MPSRCH program (Oxford Molecular). Someone of ordinary skill in the art would readily be able to determine the ideal gap open penalty and gap extension penalty for a particular nucleic acid sequence. Exemplary search parameters for use with the MPSRCH program in order to identify sequences of a desired sequence identity are as follows: gap open penalty: −16; and gap extension penalty: −4.

Degenerate variants are also encompassed by the instant invention. The degeneracy of the genetic code permits substitution of certain codons by other codons which specify the same amino acid and hence would give rise to the same protein. The nucleic acid sequence can vary substantially since, with the exception of methionine and tryptophan, the known amino acids can be coded for by more than one codon. Thus, portions or all of the markers could be synthesized to give a nucleic acid sequence significantly different from that shown in Table I. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of one or more of the markers shown in Tables I-III, or a derivative thereof. Any nucleotide or polynucleotide may be used in this regard, provided that its addition, deletion or substitution does not alter the amino acid sequence which is encoded by the nucleotide sequence. For example, the present invention is intended to include any nucleic acid sequence resulting from the addition of ATG as an initiation codon at the 5′-end of the pregnancy specific marker nucleic acid sequence or its functional derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the markers shown in Table I and fragments thereof permitted by the genetic code are, therefore, included in this invention.

Nucleic acid sequences encoding pregnancy specific markers may be isolated from appropriate biological sources using methods known in the art. In a preferred embodiment, a cDNA clone is isolated from a cDNA expression library of bovine origin. In an alternative embodiment, utilizing the sequence information provided by the cDNA sequence, genomic clones encoding a pregnancy specific marker gene may be isolated. Alternatively, cDNA or genomic clones having homology with the markers shown in Tables I-III may be isolated from other species, such as mouse or human, using oligonucleotide probes corresponding to predetermined sequences within the pregnancy specific marker gene.

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Genomic clones of the invention encoding the human or mouse pregnancy specific marker gene may be maintained in lambda phage FIX II (Stratagene).

Specific probes for identifying such sequences as the markers shown in Table I-III may be between 15 and 40 nucleotides in length.

In accordance with the present invention, nucleic acids having the appropriate level of sequence homology with the sequences encoding pregnancy specific markers may be identified by using hybridization and washing conditions of appropriate stringency as previously set forth herein.

### III. Pregnancy Specific Marker Proteins and Methods of Making the Same

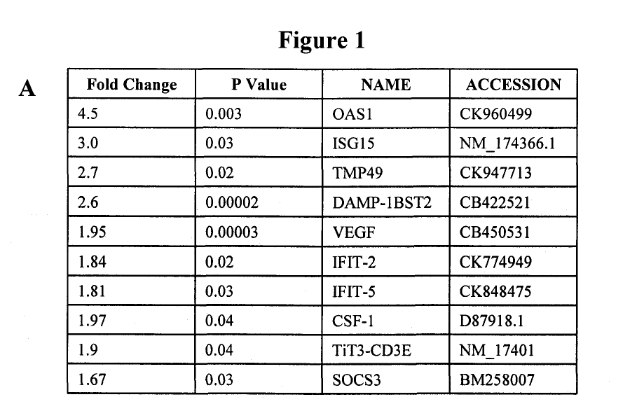

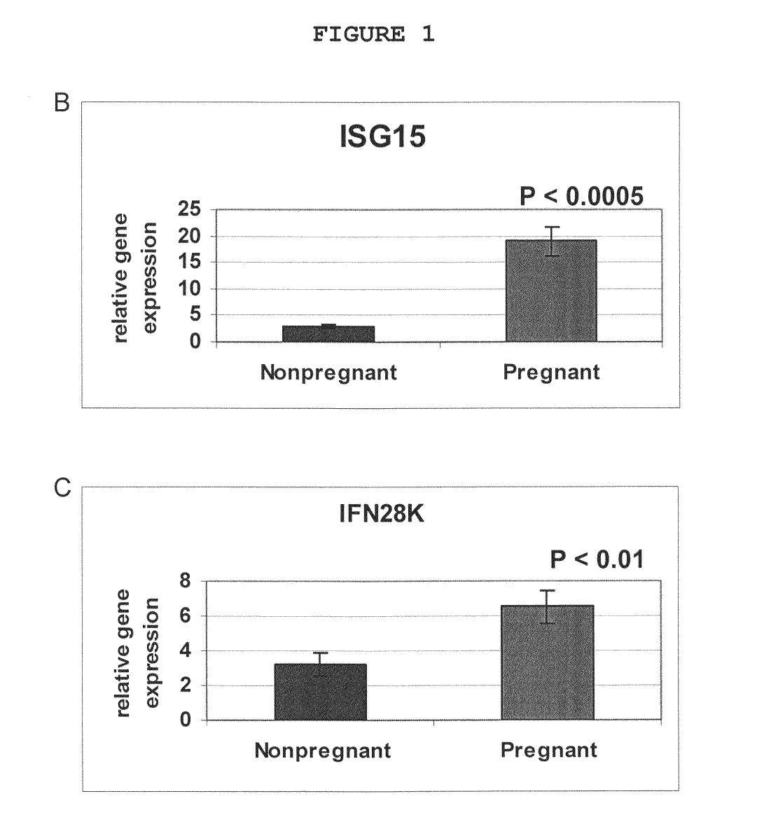

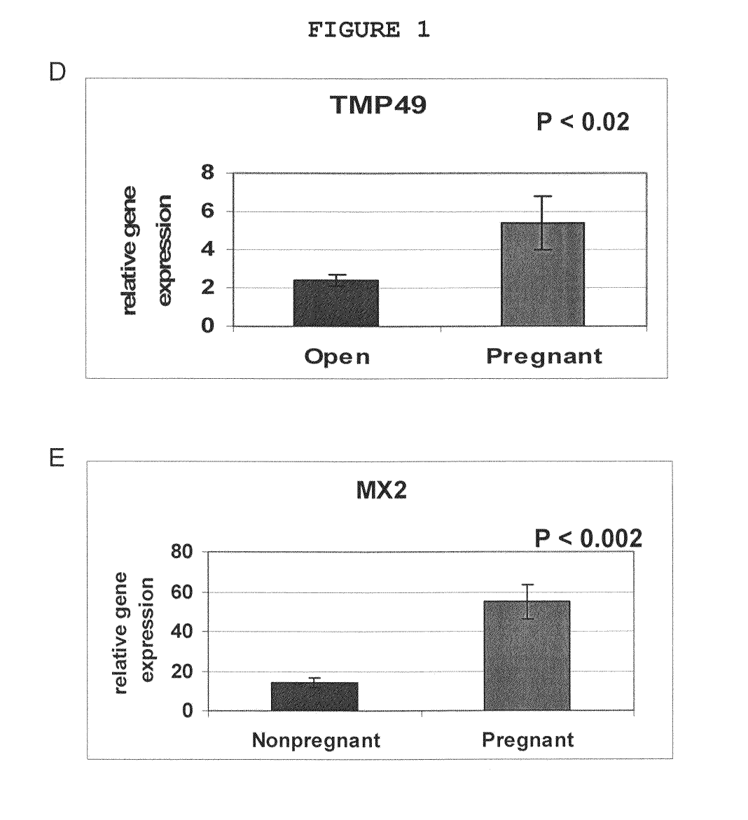

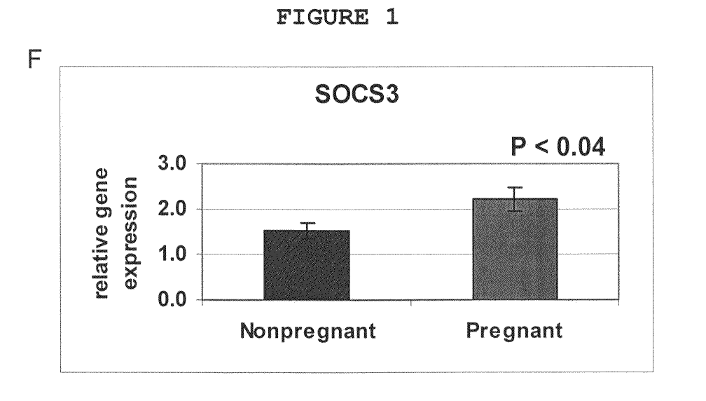

Encompassed by the invention are isolated, purified, or enriched pregnancy specific marker polypeptides, including allelic variations, analogues, fragments, derivatives, mutants, and modifications of the same which retain pregnancy specific marker function. Preferably, pregnancy specific marker polypeptides include polypeptides encoded by one or more of the sequences shown in FIG. 1A. Pregnancy specific marker function is defined above, and includes increased expression in response to pregnancy or interferon tau, or immunological cross-reactivity with an antibody reactive with the polypeptides encoded by one or more of the sequences shown in FIG. 1A, or sharing an epitope with the same (as determined for example by immunological cross-reactivity between the two polypeptides.)

Pregnancy specific marker polypeptides or proteins can be encoded by one or more of the sequences shown in FIG. 1A, and further may include variants which are at least about 75%, or 80% or 85% or 90% or 95%, and often, more than 90%, or more than 95% homologous to the same over the full length sequence. Pregnancy specific marker polypeptides also may be 60% or 65% or 70% or 75% or 80% or 85% or 90% or 95% or 97% or 98% or 99% or greater than 99% homologous to polypeptides encoded by one or more of the sequences shown in FIG. 1A over the full length sequence. All homology may be computed by algorithms known in the art, such as BLAST, described in Altschul et al. (1990), J. Mol. Biol. 215:403-10, or the Smith-Waterman homology search algorithm as implemented in MPSRCH program (Oxford Molecular). Someone of ordinary skill in the art would readily be able to determine the ideal gap open penalty and gap extension penalty for a particular protein sequence. Exemplary search parameters for use with the MPSRCH program in order to identify sequences of a desired sequence identity are as follows: gap open penalty: −12; and gap extension penalty: −2.

A full-length or truncated pregnancy specific marker protein of the present invention may be prepared in a variety of ways, according to known methods. The protein may be purified from appropriate sources, e.g., transformed bacterial or animal cultured cells or tissues, by immunoaffinity purification. Additionally, the availability of nucleic acid molecules encoding pregnancy specific markers enables production of the protein using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of full length or truncated pregnancy specific marker polypeptides may be produced by expression in a suitable prokaryotic or eukaryotic system. For example, part or all of a DNA molecule, such as one or more of the sequences shown in FIG. 1A, may be inserted into a plasmid vector adapted for expression in a bacterial cell, such as E. coli. Such vectors comprise the regulatory elements necessary for expression of the DNA in the host cell (e.g. E. coli) positioned in such a manner as to permit expression of the DNA in the host cell. Such regulatory elements required for expression include promoter sequences, transcription initiation sequences and, optionally, enhancer sequences.

The pregnancy specific marker produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

The pregnancy specific marker proteins of the invention, prepared by the aforementioned methods, may be analyzed according to standard procedures. For example, such proteins may be subjected to amino acid sequence analysis, according to known methods.

The method for making bovine pregnancy specific marker antibodies comprises providing a polypeptide of one of the foregoing amino acid sequences, administering the peptide to a mammal under conditions appropriate for stimulation of an immune response; and either isolating a polyclonal antibody from the mammal, the polyclonal antibody being capable of binding to a selected polypeptide, or isolating antibody-producing cells from the mammal, fusing the antibody producing cells with immortalizing cells to produce a hybridoma cell line, and screening the resulting hybridoma cell line to identify a cell line secreting a monoclonal antibody having a desired specificity. Other methods of making antibodies known in the art can be used such as (Harlow and Lane (1988) Antibodies: A Laboratory Manual, Cold Spring Harbor Laboratories; Goding (1986) Monoclonal Antibodies: Principles and Practice, 2d ed., Academic Press, New York; Ausubel et al. (1993) Current Protocols in Molecular Biology, Wiley Interscience/Greene Publishing, New York, N.Y; and US Patent Application No: 2003/0224452).

### IV. Methods of Using Pregnancy Specific Marker Polynucleotides, Polypeptides, and Antibodies for Pregnancy Detection Assays

Pregnancy specific marker nucleic acids, including but not limited to those listed in Tables I-III, may be used for a variety of purposes in accordance with the present invention. Pregnancy specific marker DNA, RNA, or fragments thereof may be used as probes to detect the presence of and/or expression of pregnancy specific markers. Methods in which pregnancy specific marker nucleic acids may be utilized as probes for such assays include, but are not limited to: (1) in situ hybridization; (2) Southern hybridization (3) northern hybridization; and (4) assorted amplification reactions such as polymerase chain reactions (PCR).

The pregnancy specific marker nucleic acids of the invention may also be utilized as probes to identify related genes from other animal species. As is well known in the art, hybridization stringencies may be adjusted to allow hybridization of nucleic acid probes with complementary sequences of varying degrees of homology. Thus, pregnancy specific marker nucleic acids may be used to advantage to identify and characterize other genes of varying degrees of relation to pregnancy specific markers, thereby enabling further characterization of pregnancy markers. Additionally, they may be used to identify genes encoding proteins that interact with pregnancy specific markers (e.g., by the “interaction trap” technique), which should further accelerate identification of the components involved in pregnancy. Finally, they may be used in assay methods to detect bovine pregnancy.

Further, assays for detecting and quantitating pregnancy specific markers, or to detect bovine pregnancy by detecting upregulation or down regulation of pregnancy specific markers may be conducted on any type of biological sample where upregulation or down regulation of these molecules is observed, including but not limited to body fluids (including blood), any type of cell (such as white blood cells, uterine cells, or endometrial cells), or body tissue (such as uterine, endometrial, or any other tissue).

From the foregoing discussion, it can be seen that pregnancy specific marker nucleic acids, pregnancy specific marker expressing vectors, pregnancy specific marker proteins and anti-pregnancy specific marker antibodies of the invention can be used to detect pregnancy specific marker expression in body tissue, cells, or fluid, and alter pregnancy specific marker protein expression for purposes of assessing the genetic and protein interactions involved in pregnancy and induced expression.

In most embodiments for screening for specific marker expression associated with pregnancy, the pregnancy specific marker nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art. Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Thus any of the aforementioned techniques may be used to detect or quantify pregnancy specific marker expression and accordingly, detect bovine pregnancy.

### V. Assays for Determining Bovine Pregnancy Utilizing the Pregnancy Specific Marker Associated Molecules of the Invention

In accordance with the present invention, it has been discovered that bovine pregnancy is correlated with altered expression levels of certain markers, including but not limited to, differentially expressed mRNAs and proteins. Thus, these molecules may be utilized in conventional assays to detect bovine pregnancies.

In an exemplary method, a blood sample is obtained from a bovine suspected of being pregnant. Optionally, the blood may be centrifuged through a Hypaque gradient to obtain the buffy coat. The blood or buffy coat preparation is diluted and optionally subjected to polymerase chain reaction conditions suitable for amplification of the pregnancy specific marker encoding mRNA. In certain applications, it may be necessary to include an agent which lyses cells prior to performing the PCR. Such agents are well known to the skilled artisan. The reaction products are then run on a gel. An alteration in pregnancy specific marker mRNA levels relative to levels obtained from a non-pregnant bovine is indicative of pregnancy in the animal being tested.

In an alternative method, uterine tissue or a chorionic villi sample is obtained from the bovine suspected of being pregnant. The cells are then lysed and PCR performed. As above, an increase in pregnancy specific marker mRNA expression levels relative to those observed in a non-pregnant animal being indicative of pregnancy in the test animal.

It is also possible to detect bovine pregnancy using immunoassays. In an exemplary method, blood is obtained from a bovine suspected of being pregnant. As above, the blood may optionally be centrifuged through a Hypaque gradient to obtain a buffy coat. The blood or buffy coat sample is diluted and at least one antibody immunologically specific for pregnancy specific markers is added to the sample. In a preferred embodiment, the antibody is operably linked to a detectable label. Also as described above, the cells may optionally be lysed prior to contacting the sample with the antibodies immunologically specific for pregnancy specific markers. Increased production of pregnancy specific markers is assessed as a function of an increase in the detectable label relative to that obtained in parallel assays using blood from a non-pregnant cow. In yet another embodiment, the blood or buffy coat preparation is serially diluted and aliquots added to a solid support. Suitable solid supports include multi-well culture dishes, blots, filter paper, and cartridges. The solid support is then contacted with the detectably labeled antibody and the amount of pregnancy specific marker protein (e.g., a protein encoded by a nucleic acid of Table I) in the animal suspected of being pregnant is compared with the amount obtained from a non-pregnant animal as a function of detectably labeled antibody binding. An increase in the pregnancy specific marker protein level in the test animal relative to the non-pregnant control animal is indicative of pregnancy.

In an alternative method, a blood sample is obtained from a bovine suspected of being pregnant. RNA can be isolated from these samples and directly hybridized to a GeneChip (Affymetrics) Bovine Genome Array (part # 900561) and is utilized in accordance with the manufacturers instructions. Product information can be found at the manufacturers website on the world-wide-web at affymetrix.com/products/arrays/specific/bovine.affx. The bovine samples can also be probed directly as methods which do not require amplification may be more amenable to quantitative analysis in certain situations. Traditional methods of direct detection well-known in the art include Northern and Southern blotting and RNase protection assays. Also as described above, Resonance Light Scattering and planar wave guide technologies allow detection of nucleic acids on microarrays without amplification of the target or signal. In yet another embodiment, the blood is collected in Tempus (Applied Biosystems) collection tubes. Whole blood specimens collected can be immediately lysed so that RNA is stabilized and stored without causing changes to the expression profile of the sample.

The foregoing immunoassay methods may also be applied to a urine sample.

### VI. Kits and Articles of Manufacture

Any of the aforementioned products can be incorporated into a kit which may contain an pregnancy specific marker polynucleotide or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, or any combination thereof.

Exemplary kits contain reagents for an immunoassay such as an ELISA (e.g., detectably labeled pregnancy specific marker antibody, solid support, multiwell dish, buffer). Such a kit may optionally further comprise reagents suitable for performing polymerase chain reaction (e.g. polymerase, agarose gel, buffer, nucleotides).

The following three sequences represent one of the preferred Bovine pregnancy inducible marker molecules:

SEQ ID NO: 1

| | |
|---|---|
| 1 | gctactattt ttgttattat tgacaataaa ttggaccatc |
| | acctttcccc tcaggttcac |
| 61 | agaaaaaagt caaggctggg attccactct tttgcctgag |
| | tctctcctgg tggctcaaaa |
| 121 | aggctattgt caggctccaa tggggagcat tgtctatgaa |
| | agaaaagaca gcacaaaata |
| 181 | acaggaaggc tgggggtcag gaaacctgga ttctggtccc |
| | aggtctgaca tcaattcaca |
| 241 | gctgtctttg gatccaagcc ccctagccct aaaagtgaaa |
| | agggctggcc caaacggctc |
| 301 | tggagacctt gcctccaaga tactctggcg ttcaaaggat |
| | ggtacatgtc cagttctctt |
| 361 | ccacctgggg agaggctcct ccgtggaact ggattcctgg |
| | agagggtcta tagtgctgtc |
| 421 | taaaatcata ggcctggaac atcaggtcac tgtgttcttg |
| | gggcgacaca tcccaggagc |
| 481 | ccacgggaga cccatcccat ttcttaaagc acgggtaatc |
| | cagccagacc aaagccgctc |
| 541 | gtgcaagccg ctcccagcta tatgcgtctt taccagcaac |
| | atttcctgta gggtccgccg |
| 601 | ggtccagaat cacaggcctg ggttttgcaa gttgctt |

Below is a sequence which is highly homologous to Genbank Accession # NM_{—}001040606.1, now

SEQ ID NO: 2

| | |
|---|---|
| 1 | gcacgagcac agattcaggc agcagctctg ccgcctctgg |
| | ctctccagtc cccagcaccg |
| 61 | tgatggagct cagaaatacc ccggccgggt ctctagacaa |
| | gttcatcgaa gaccacctcc |
| 121 | tgccagacga ggagttccgc atgcaggtca aagaagccat |
| | cgacatcatc tgcactttcc |
| 181 | tgaaggagag gtgtttccga tgtgcccctc acagagttcg |
| | ggtgtccaaa gttgtgaagg |
| 241 | gcggctcctc aggcaaaggc acgaccctca ggggacgatc |
| | agatgctgac ctcgtcgtct |
| 301 | tcctcaccaa tctcacaagt tttcaggaac agcttgagcg |
| | ccgaggagaa ttcatcgaag |
| 361 | aaatcaggag acagctggaa gcctgtcaaa gagaggaaac |
| | atttgaagtg aagtttgagg |
| 421 | tccagaaacg gcaatgggag aatccccgcg ctctcagctt |
| | tgtgctgagg tcccccaagc |
| 481 | tcaaccaggc ggtggagttc gatgtcctgc ccgcctttga |
| | tgccctaggt cagttgacca |
| 541 | aaggttacag acctgactct agagtctatg tccggctcat |
| | ccaagagtgc aagtacctga |
| 601 | agagagaagg cgagttctcc ccctgcttca cggagctgca |
| | gcgagacttc ctgaagaatc |
| 661 | gtccaaccaa gctgaagagc ctcatccgcc tggtgaagca |
| | ctggtaccaa ctgtgtaagg |
| 721 | agcagcttgg aaagccattg cccccacaat atgctctgga |
| | gcttctgacg gtctatgcct |
| 781 | gggagcaagg atgcaataaa acaggattca tcacagctca |
| | gggatttcag actgtcttga |
| 841 | aattagtcct aaagtatcag aagctttgca tctactggga |
| | aaagaactat aactctgaaa |
| 901 | accctattat tgaagaatat ctgacgaagc aacttgcaaa |
| | acccaggcct gtgattctgg |
| 961 | acccggcgga ccctacagga aatgttgctg gtaaagacgc |
| | aaatagctgg gagcggcttg |
| 1021 | cacaagcggc tttggtctgg ctggattacc cgtgctttaa |
| | gaaatgggat gggtctcccg |
| 1081 | tgggctcctg ggatgtgtcg ccccaagaac acagtgacct |
| | gatgttccag gcctatgatt |
| 1141 | ttagacagca ctgtagaccc tctccaggaa tccagttcca |
| | cggaggagcc tctccccagg |
| 1201 | tggaagagaa ctggacatgt accatcctct gaatgccaga |
| | gtatcttgga ggcaaggtct |
| 1261 | ccagagccgt ctgggccagc cctcttcact tctagggata |
| | gggggcttgg atccaaagac |
| 1321 | agctgtgaat tgatgtcaga cctgggacca gaatccaggt |
| | ctcctgaccc ccagccttcc |
| 1381 | tgctattctg tgctgtcttt tctttcatag acaatgctcc |
| | ccattggagc ctgacaatag |
| 1441 | cctctctgag ccaccaggag agactcaggc aaaagagtgg |
| | aatcccagcc ttgactttct |
| 1501 | tctgtgaacc tgaggggaaa ggtgatggtc caatttattg |
| | tcaataataa caaaaatagt |
| 1561 | agcaaatgcc atttgttggg tgttaattag cttcaaggta |
| | cagcgccaag aagtatacct |
| 1621 | gcatattatg tgtgtgtgtg catattcatt gattcaacta |
| | aagatattaa ttgggcacct |
| 1681 | gc |

The amino acid sequence for SEQ ID NO: 2 is SEQ ID NO: 3:

SEQ ID NO: 3:
MELRNTPAGSLDKFIEDHLLPDEEFRMQVKEAIDIICTFLKERCFRCAPH
RVRVSKVVKGGSSGKGTTLRGRSDADLVVFLTNLTSFQEQLERRGEFIEE
IRRQLEACQREETFEVKFEVQKRQWENPRALSFVLRSPKLNQAVEFDVLP
AFDALGQLTKGYRPDSRVYVRLIQECKYLKREGEFSPCFTELQRDFLKNR
PTKLKSLIRLVKHWYQLCKEQLGKPLPPQYALELLTVYAWEQGCNKTGFI
TAQGFQTVLKLVLKYQKLCIYWEKNYNSENPIIEEYLTKQLAKPRPVILD
PADPTGNVAGKDANSWERLAQAALVWLDYPCFKKWDGSPVGSWDVSPQEH
SDLMFQAYDFRQHCRPSPGIQFHGGASPQVEENWTCTIL

The following sequence represents another of the preferred Bovine pregnancy inducible marker molecules:

SEQ ID NO: 4:

| | |
|---|---|
| 1 | tccagcggcc gcggctcgag ctttgtcctg gctcccaatc |
| | ccttccggca gaaagtgcag |
| 61 | ggctgggact cacacttgga tgagcagact tgggacagat |
| | ctgtgtcacc ttgtcctgcc |
| 121 | acctccatcc cccaagctcc tattcatccc acagtaccca |
| | gctctgggag cccctcccca |
| 181 | gagacttctc tgcttctcaa aatgaagtcc tcgaagagtt |
| | ctctgatgac ttggtggtta |
| 241 | ggattccagg ctcccgtggc ctgggttcag tccctggtct |
| | aggaactaat atcccataag |
| 301 | ctgggagaca tgaccagtaa aaaggaaaga acgctctccc |
| | tgtgtgtgcc ccctgctcct |
| 361 | aaaatggtgt ttgatggtgg cttttgttaa ctgagcccct |
| | tataagcgta tggtttctac |
| 421 | atgactacaa agaatgttcc cgaatgcttt actggaatga |
| | agcttgtgaa aaataacaaa |
| 481 | gagccaaagt ggaaactgtt tgagtgacaa aataaatatc |
| | atcacgtgta aaataaatat |
| 541 | ccatgaattc gcaggaatat aaatacatga ctgactaaaa |
| | caataaatga aagagaggag |
| 601 | aaaaatctcc caggccgaaa aa |

### EXAMPLES

The following examples are included to illustrate certain embodiments of the invention. They are not intended to limit the invention in any way.

### Example I

### Methods for Tissue and Blood Collection and Subsequent RNA Isolation

For Endometrial Tissue, uteri of day 18 pregnant or non-pregnant cows were removed surgically by hemi-hysterectomy. The Ispilateral uterine horn was cut open lengthwise to expose the endometrium (the layer lining the uterine lumen) and the endometrium was stripped using scissors and forceps. The endometrial tissue was snap frozen in dry ice and then stored at −80° C. until RNA extraction was performed later. Endometrial tissue was collected in this manner for 3 non-pregnant and 3 pregnant cows (i.e. n=3). For endometrial RNA extraction, 100 mg of frozen tissue was placed in 1 ml of TRI reagent (Sigma Chemical Co.; St. Louis, Mo.) and homogenized using an electronic tissue grinder (IKA Laboratories; Wilmington, N.C.) for 30 seconds, maximum speed. The homogenate was allowed to sit at room temperature for 5 minutes then 0.2 ml of chloroform was added with shaking and the homogenate was incubated at room temperature for another 10 minutes. The homogenate was then centrifuged for 15 minutes, 13,000 g and 4° C. The upper aqueous layer was removed and placed in a new tube containing 0.5 ml of isopropanol, mixed and incubated at room temperature for 10 minutes. The RNA was precipitated by centrifuging for 10 minutes, 12,000 g and 4° C. The RNA pellet was washed once with 70% ethanol, centrifuged for 5 minutes, 12,000 g and dried using a speed vac concentrator. The RNA pellet was resuspended in 0.030 ml RNase free water and quantitated by UV spectrophotometry using an absorbance of 280 nm. Ten micrograms of RNA was sent on dry ice via FEDX to the University of Colorado Health Sciences Center—DNA Micro Array Core laboratory for Gene Chip screening.

For blood RNA, Blood samples were collected using vacutainers (Becton-Dickinson and Company; Franklin Lakes, N.J.); containing potassium EDTA (ethylene-diamine-tetra-acetic acid) and was processed using QIAamp procedures and reagents from Qiagen Inc.; Valencia, Calif.) as follows. Blood was aliquotted into 15 ml conical tubes-1 ml per tube, three tubes per sample and 5 ml erythrocyte lysis buffer was added to each tube. Samples were incubated on ice for 20 minutes then centrifuged for 10 minutes, 1500 rpm at 4° C. The white cell pellets were washed with 2 ml erythrocyte lysis buffer and centrifuged again to get rid of red blood cell contaminants. Supernatants were discarded and the white cell pellets were resuspended in RLT lysis buffer (from the same Qiagen kit) containing 1% beta-Mercaptoethanol and frozen on dry ice for transport back to our lab. These lysates were thawed at 37° C. for 10 minutes. The thawed lysate was then pipetted into a QIAshredder spin column and centrifuged for 2 minutes at maximum speed in a microcentrifuge at room temperature. Then 70% ethanol was added to the lysate and the mixture was pipetted into a new spin column and centrifuged for 15 seconds at 10,000 rpm, room temperature. The flow through was discarded and the RNA was treated with DNase I (27.3 units) by applying DNase I directly to the column containing bound RNA. The column was allowed to sit at room temperature for 15 minutes and then washed once with buffer RW1 and twice with buffer RPE (both supplied by the kit) by adding buffer and centrifuging at 10,000 rpm for 15 seconds. Each time the flow through was discarded. The column was centrifuged one final time to ensure removal of all buffers prior to elution, then the column was placed into a new collection tube and the RNA was eluted with 0.030 ml RNase free water also supplied by the kit and a final centrifugation at 10,000 rpm for 1 minute. Triplicate sets of samples were pooled into one final sample such that there was one sample from each pregnant (n=3) or non-pregnant (n=3) cows. These samples were packaged in dry ice and sent via FEDX to the University of Colorado Health Sciences—DNA micro Array core facility for Gene Chip screening. Gene chips were purchased from Affymetrix and shipped directly to UCHSC for screening. The results are shown in Tables I-III below.

FIG. 1A provides preferred bovine pregnancy markers identified in accordance with the present invention. The fold change in expression levels in pregnant cows when compared to non pregnant cows, the P Value, an abbreviated name and the NCBI Accession number are shown. A complete description of the nucleotide sequence and other background information can be found on the NCBI website.

The ISG15 and MX2 targets are employed as positive controls and have been previously described. FIGS. 1B-1F represents Real Time PCR confirmation of several of the clones listed in FIG. 1A. Three day 18 pregnant and three day 18 non-pregnant cows are represented in this analysis using specific oligonucleotide primers for each target.

In more recent experiments, blood from a fourth pregnant cow (day 18) was assessed using a more refined statistical analysis and normalization approach. When using blood cell mRNA from three non-pregnant cows and four pregnant cows in the microarray analysis, additional targets were identified. These clones have been sorted based on fold change. See Table 1 and 2. Note that this analysis provides the rank order out of the 23,000 genes that were identified. We have learned over the past year that the most significant fold changes actually translate into confirmed targets when using Real time PCR. Preferred targets have fold changes greater than 1.8 and P values less than 0.01. Also, note that we now have highlighted two down regulated targets in addition to the upregulated targets. We have other analyses that are similar over the past year, but had settled on the enclosed list. This analysis was completed on Sep. 14, 2006.

TABLE I
BLOOD - INCREASE

| No. | Ratio | p-value | Identifier | Gene Name |
|---|---|---|---|---|
| 1 | 5.00 | 0.00507 | CK960499 | 2′-5′-oligoadenylate synthetase 1 (OAS1) |
| 2 | 3.11 | 0.00043 | AW658522 | Transcribed locus, strongly similar to NP_859075.1 hypothetical protein LOC33877 [symbol] |
| 3 | 3.08 | 0.00952 | NM_174366 | interferon-stimulated protein, 15 kDa |
| 4 | 2.97 | 0.00602 | CK947713 | Transcribed locus, strongly similar to XP_511922.1 similar to hypothetical prote [symbol] |
| 5 | 2.63 | 0.04296 | CB530781 | Transcribed locus |
| 6 | 2.51 | 0.02204 | CK943256 | Transcribed locus |
| 7 | 2.50 | 0.04602 | AJ006574 | T-cell receptor beta chain variable segment, clone C55 |
| 8 | 2.49 | 0.03502 | U73187 | T cell receptor gamma chain variable region BVG3.2 |
| 9 | 2.49 | 0.01077 | CK846889 | Transcribed locus, weakly similar to NP_004326.1 bone marrow stromal cell antige [symbol] |
| 10 | 2.46 | 0.04679 | CK973287 | Transcribed locus, weakly similar to XP_526999.1 fibrillin 2 (congenital contrac [symbol] |
| 11 | 2.42 | 0.02983 | CB426512 | Transcribed locus |
| 12 | 2.34 | 0.04915 | BP109430 | Transcribed locus, strongly similar to NP_065173.2 prostaglandin F2 receptor neg [symbol] |
| 13 | 2.31 | 0.02461 | CB439500 | Transcribed locus, moderately similar to NP_055864.1 amyotrophic lateral scleros [symbol] |
| 14 | 2.28 | 0.01671 | NM_173895 | bactericidal/permeability-increasing protein |
| 15 | 2.26 | 0.04778 | NM_173941 | myxovirus (influenza virus) resistance 2 (mouse) |
| 16 | 2.10 | 0.03064 | BM446374 | Transcribed locus |
| 17 | 2.10 | 0.03256 | CB456207 | Transcribed locus, strongly similar to NP_002779.1 proteasome (prosome, macropai [symbol] |
| 18 | 2.08 | 0.00576 | AW659977 | T cell receptor, beta cluster |
| 19 | 2.07 | 0.01832 | AB008616 | MHC class I heavy chain, partial cds, clone P5647.6m |
| 20 | 2.07 | 0.02382 | BI848417 | Transcribed locus, strongly similar to NP_003452.1 zyxin [Homo sapiens] |
| 21 | 2.03 | 0.02886 | CB461274 | component 3 |
| 22 | 2.02 | 0.00947 | CB422521 | Transcribed locus, weakly similar to NP_004326.1 bone marrow stromal cell antige [symbol] |
| 23 | 2.00 | 0.04554 | CK846547 | Transcribed locus |
| 24 | 1.97 | 0.00569 | CK966909 | Transcribed locus, moderately similar to NP_003326.2 ubiquitin-activating enzyme [symbol] |
| 25 | 1.96 | 0.02027 | AW464305 | Transcribed locus, strongly similar to NP_084520.2 immediate early response 5-li [symbol] |
| 26 | 1.96 | 0.02170 | CB433489 | Transcribed sequence with weak similarity to protein pir: S48218 (H. sapiens) S482 [symbol] |
| 27 | 1.96 | 0.03922 | AF127029 | uncoupling protein 2 (mitochondrial, proton carrier) |
| 28 | 1.95 | 0.01739 | CK777675 | Transcribed locus, moderately similar to XP_513514.1 similar to Interferon-induc [symbol] |
| 29 | 1.94 | 0.02759 | CK951386 | Transcribed locus, strongly similar to NP_003452.1 zyxin [Homo sapiens] |
| 30 | 1.94 | 0.03380 | CK769989 | Transcribed locus, strongly similar to NP_071886.1 activin A receptor type II-li [symbol] |
| 31 | 1.93 | 0.00367 | NM_174011 | antigen CD3E, epsilon polypeptide (TiT3 complex) |
| 32 | 1.93 | 0.00492 | CB420023 | Transcribed sequence |
| 33 | 1.92 | 0.03670 | CB452278 | Transcribed locus, moderately similar to NP_002277.3 lymphocyte-activation gene [symbol] |
| 34 | 1.92 | 0.04748 | CK955157 | Transcribed locus, moderately similar to XP_513514.1 similar to Interferon-induc [symbol] |
| 35 | 1.92 | 0.04989 | BM288577 | Transcribed locus, strongly similar to NP_002779.1 proteasome (prosome, macropai [symbol] |
| 36 | 1.92 | 0.00804 | CB447603 | Transcribed locus, strongly similar to NP_035247.1 plectin 1 [Mus musculus] |
| 37 | 1.91 | 0.04585 | CK846935 | Transcribed locus, moderately similar to NP_077024.1 likely ortholog of mouse D1 [symbol] |
| 38 | 1.91 | 0.02124 | AU098038 | Transcribed locus, strongly similar to NP_005310.1 histone 1, H1c [Homo sapiens] [symbol] |
| 39 | 1.90 | 0.04041 | CB420649 | Transcribed locus |
| 40 | 1.88 | 0.02302 | CK775650 | Transcribed locus |
| 41 | 1.88 | 0.02907 | CB533299 | Transcribed locus |
| 42 | 1.88 | 0.01044 | CB420282 | Transcribed sequence with weak similarity to protein ref: NP_054886.1 (H. sapiens) [symbol] |
| 43 | 1.87 | 0.03536 | CB441353 | Transcribed locus, weakly similar to NP_004891.3 apolipoprotein B mRNA editing e [symbol] |
| 44 | 1.85 | 0.04171 | CK849539 | Transcribed locus, moderately similar to NP_033665.1 lectin, galactoside-binding [symbol] |
| 45 | 1.85 | 0.00171 | CK943316 | Transcribed locus, strongly similar to NP_031958.1 endoglin [Mus musculus] [symbol] |
| 46 | 1.85 | 0.04295 | CB431728 | Transcribed sequences |
| 47 | 1.83 | 0.00772 | CB432365 | Transcribed locus, moderately similar to XP_520524.1 similar to DEAD/H (Asp-Glu- [symbol] |
| 48 | 1.82 | 0.04414 | CB450531 | Vascular endothelial growth factor |
| 49 | 1.81 | 0.01211 | CK774949 | Transcribed locus, moderately similar to NP_001538.3 interferon-induced protein [symbol] |
| 50 | 1.80 | 0.03032 | BM251565 | Transcribed locus, moderately similar to XP_290768.4 chromosome 17 open reading [symbol] |
| 51 | 1.80 | 0.03019 | BP108674 | Transcribed locus, strongly similar to NP_112552.1 heterogeneous nuclear ribonuc [symbol] |
| 52 | 1.79 | 0.03770 | CK945023 | Antigen CD3D, delta polypeptide (TiT3 complex) |
| 53 | 1.79 | 0.00569 | CK977771 | Transcribed locus, strongly similar to XP_524929.1 LOC469546 [Pan troglodytes] |
| 54 | 1.78 | 0.01998 | NM_174059 | frizzled-related protein |
| 55 | 1.78 | 0.04464 | CK947663 | Transcribed locus |
| 56 | 1.77 | 0.01756 | NM_174641 | guanylate cyclase 1, soluble, beta 3 |
| 57 | 1.77 | 0.01367 | BM251221 | Transcribed locus |
| 58 | 1.77 | 0.01394 | BE667175 | Transcribed locus |
| 59 | 1.77 | 0.01461 | CB424466 | Transcribed locus, strongly similar to NP_003864.2 neuropilin 1 [Homo sapiens] |
| 60 | 1.77 | 0.01279 | CK848475 | Transcribed locus, strongly similar to XP_521554.1 interferon-induced protein wi [symbol] |
| 61 | 1.76 | 0.01340 | NM_178109 | protein kinase, interferon-inducible double stranded RNA dependent |
| 62 | 1.75 | 0.04955 | CB460423 | Transcribed sequence |
| 63 | 1.74 | 0.02503 | D90132 | T cell receptor, beta cluster |
| 64 | 1.74 | 0.01945 | CK955838 | Transcribed locus |
| 65 | 1.74 | 0.02281 | CK963576 | Transcribed locus, moderately similar to NP_056616.1 neuropathy target esterase [symbol] |
| 66 | 1.74 | 0.03401 | BF601200 | Transcribed locus, moderately similar to XP_515189.1 similar to Rho GTPase activ [symbol] |
| 67 | 1.72 | 0.02595 | CK942526 | Transcribed locus |
| 68 | 1.72 | 0.04491 | CK771931 | Transcribed locus, moderately similar to XP_284175.3 RIKEN cDNA 1110055E19 |
| | | | | gene |
| 69 | 1.72 | 0.02786 | CK941897 | Transcribed locus, strongly similar to NP_032538.1 low density lipoprotein recep [symbol] |
| 70 | 1.70 | 0.00976 | BI681282 | Integrin, alpha L (antigen CD11A (p180), lymphocyte function-associated antigen [symbol] |
| 71 | 1.70 | 0.02745 | CK848995 | Transcribed locus |
| 72 | 1.70 | 0.04812 | CB533126 | Transcribed locus, strongly similar to NP_000169.1 glutathione synthetase [Homo [symbol] |
| 73 | 1.70 | 0.03253 | BF045590 | Transcribed locus, strongly similar to XP_516581.1 similar to death effector dom [symbol] |
| 74 | 1.69 | 0.04126 | CB439389 | Transcribed locus, strongly similar to NP_852070.1 arginyl aminopeptidase (amino [symbol] |
| 75 | 1.68 | 0.03073 | AW345246 | Transcribed locus |
| 76 | 1.67 | 0.00470 | CB171752 | Transcribed locus, strongly similar to NP_006280.2 talin 1 [Homo sapiens] |
| 77 | 1.67 | 0.00777 | CB450623 | Transcribed locus, strongly similar to XP_514712.1 LOC458321 [Pan troglodytes] |
| 78 | 1.67 | 0.01644 | CB443461 | Transcribed locus, weakly similar to XP_219476.1 similar to interferon-induced p [symbol] |
| 79 | 1.66 | 0.00374 | AB042274 | CD6 antigen |
| 80 | 1.66 | 0.01685 | NM_198221 | integrin, alpha L (antigen CD11A (p180), lymphocyte function-associated antigen [symbol] |
| 81 | 1.66 | 0.04590 | CK769666 | Transcribed locus, moderately similar to XP_508072.1 similar to hypothetical pro [symbol] |
| 82 | 1.66 | 0.02013 | BP111432 | Transcribed locus, strongly similar to XP_515320.1 similar to rhoB gene [Pan tro [symbol] |
| 83 | 1.65 | 0.04049 | CK945847 | Integrin, alpha 5 (fibronectin receptor, alpha polypeptide) |
| 84 | 1.64 | 0.00669 | CK942999 | Transcribed locus, strongly similar to XP_514712.1 LOC458321 [Pan troglodytes] |
| 85 | 1.64 | 0.00398 | BM031140 | Transcribed sequence |
| 86 | 1.63 | 0.00699 | BE589764 | filamin A, alpha (actin binding protein 280) |
| 87 | 1.63 | 0.04775 | CK847695 | Transcribed locus |
| 88 | 1.63 | 0.02775 | CB437938 | Transcribed locus |
| 89 | 1.61 | 0.04583 | NM_173972 | xanthene dehydrogenase |
| 90 | 1.60 | 0.04844 | CB172009 | T cell receptor, beta cluster |
| 91 | 1.60 | 0.01663 | CB429901 | Transcribed locus |
| 92 | 1.60 | 0.04279 | CK769972 | Transcribed locus |
| 93 | 1.60 | 0.04845 | AW482092 | Transcribed locus, moderately similar to NP_001965.3 egf-like module containing, [symbol] |
| 94 | 1.60 | 0.04922 | CB432608 | Transcribed locus, strongly similar to NP_005345.2 jun D proto-oncogene [Homo sa [symbol] |
| 95 | 1.60 | 0.01168 | CK970094 | Transcribed locus, strongly similar to NP_872270.1 pyruvate kinase, muscle [Homo [symbol] |
| 96 | 1.60 | 0.01530 | CK950619 | Transcribed locus, strongly similar to XP_510898.1 similar to KIAA0370 [Pan trog [symbol] |
| 97 | 1.60 | 0.03449 | CK770077 | Transcribed sequences |
| 98 | 1.59 | 0.04056 | CK727467 | Transcribed locus |
| 99 | 1.59 | 0.01219 | BE723335 | Transcribed locus, moderately similar to NP_001563.2 interferon regulatory facto [symbol] |
| 100 | 1.59 | 0.00476 | CB461321 | Transcribed locus, weakly similar to XP_513247.1 interferon, alpha-inducible pro [symbol] |

[symbol]

indicates data missing or illegible when filed

TABLE II
BLOOD - DECREASE

| No. | Ratio | p-value | Identifier | Gene Name |
|---|---|---|---|---|
| 1 | 5.42 | 0.03062 | BF040394 | Transcribed locus, weakly similar to XP_530423.1 LOC459039 [Pan |
| | | | | troglodytes] |
| 2 | 4.21 | 0.02614 | CB430090 | Transcribed locus, weakly similar to XP_530423.1 LOC459039 [Pan |
| | | | | troglodytes] |
| 3 | 2.79 | 0.03457 | CB430859 | Transcribed locus, strongly similar to XP_523901.1 UDP-Gal: betaGlcNAc beta |
| | | | | 1,4- [symbol] |
| 4 | 2.48 | 0.03336 | BP109170 | Transcribed locus |
| 5 | 2.36 | 0.04649 | CB420502 | Transcribed locus, strongly similar to NP_940926.1 testis expressed gene 9 |
| | | | | [Homo [symbol] |
| 6 | 2.32 | 0.04224 | CK951818 | Transcribed locus |
| 7 | 2.21 | 0.03663 | CK775610 | Transcribed locus, strongly similar to XP_510553.1 similar to homer-2a [Pan |
| | | | | trog [symbol] |
| 8 | 2.13 | 0.00762 | CK848111 | Transcribed locus, strongly similar to NP_115522.1 ADP-ribosylation factor-like [symbol] |
| 9 | 2.02 | 0.04403 | BE663513 | Transcribed sequence with moderate similarity to protein prf: 2206383A |
| | | | | (H. sapiens [symbol] |
| 10 | 2.01 | 0.03196 | CF762128 | Transcribed sequences |
| 11 | 2.00 | 0.03895 | CK962901 | Transcribed locus, strongly similar to NP_057916.1 B-cell CLL/lymphoma 11A |
| | | | | (zinc [symbol] |
| 12 | 2.00 | 0.02704 | CK948091 | Transcribed locus, strongly similar to XP_518548.1 leucine rich repeat |
| | | | | containin [symbol] |
| 13 | 1.98 | 0.04275 | CK956417 | Transcribed locus |
| 14 | 1.97 | 0.01409 | NM_174377 | keratin 10 (epidermolytic hyperkeratosis) |
| 15 | 1.97 | 0.02158 | AW632120 | Transcribed locus, strongly similar to XP_528098.1 frizzled 3 [Pan troglodytes] |
| 16 | 1.95 | 0.04144 | BM251983 | Transcribed locus |
| 17 | 1.93 | 0.02193 | CB166129 | Transcribed locus, moderately similar to NP_068593.2 mitochondrial ribosomal |
| | | | | pro [symbol] |
| 18 | 1.93 | 0.04918 | CK965625 | Transcribed locus, strongly similar to XP_522364.1 similar to amphoterin |
| | | | | induced [symbol] |
| 19 | 1.91 | 0.02174 | CK976667 | Transcribed locus, moderately similar to NP_068593.2 mitochondrial ribosomal |
| | | | | pro [symbol] |
| 20 | 1.90 | 0.04041 | CK847416 | Transcribed locus |
| 21 | 1.90 | 0.02978 | CB535359 | Transcribed locus, strongly similar to NP_004989.1 myosin IE [Homo sapiens] |
| 22 | 1.89 | 0.03687 | AV607578 | Transcribed locus, strongly similar to NP_060205.3 hypothetical protein |
| | | | | FLJ20272 [symbol] |
| 23 | 1.88 | 0.04562 | CB445539 | Transcribed locus, strongly similar to NP_598511.1 RIKEN cDNA 3110048E14 |
| | | | | gene [M [symbol] |
| 24 | 1.84 | 0.04470 | CK770380 | Transcribed locus |
| 25 | 1.82 | 0.03401 | CK775320 | Transcribed locus, strongly similar to NP_004285.2 mitochondrial translational r [symbol] |
| 26 | 1.82 | 0.04961 | CK775813 | Transcribed locus, strongly similar to XP_371813.2 kinesin family member C1 |
| | | | | [Hom [symbol] |
| 27 | 1.81 | 0.01186 | CK846762 | Transcribed locus |
| 28 | 1.79 | 0.03440 | NM_203362 | gb: NM_203362.1 /DB_XREF = gi: 42733601 /GEN = MTPN /TID = Bt.647.1 |
| | | | | /CNT = 10 /FEA = FLmRNA [symbol] |
| 29 | 1.79 | 0.02472 | CK849280 | Transcribed locus, moderately similar to XP_519630.1 similar to arylamine N- |
| | | | | acet [symbol] |
| 30 | 1.78 | 0.03341 | CB421393 | Transcribed locus, strongly similar to NP_789794.1 Bardet-Biedl syndrome 7 |
| | | | | [Homo [symbol] |
| 31 | 1.77 | 0.02082 | CK944284 | Transcribed locus, moderately similar to NP_766232.1 RIKEN cDNA |
| | | | | 5830468K18 gene |
| 32 | 1.77 | 0.02852 | CK848122 | Transcribed locus, strongly similar to NP_004631.1 HLA-B associated transcript 1 |
| 33 | 1.77 | 0.04067 | BE754659 | Transcribed locus, weakly similar to XP_522710.1 hypothetical protein |
| | | | | XP_522710 |
| 34 | 1.76 | 0.00218 | CK940769 | Transcribed locus, strongly similar to NP_082407.1 RIKEN cDNA 2610510J17 |
| | | | | gene [M [symbol] |
| 35 | 1.76 | 0.02278 | CB424304 | Transcribed locus, strongly similar to XP_345655.1 similar to protein similar to [symbol] |
| 36 | 1.75 | 0.00380 | CK954686 | Transcribed locus, strongly similar to NP_008973.1 ribonuclease P 14 kDa |
| | | | | subunit [symbol] |
| 37 | 1.74 | 0.01366 | CK846731 | Transcribed locus, moderately similar to NP_065104.1 apoptosis, caspase |
| | | | | activati [symbol] |
| 38 | 1.73 | 0.00738 | AV667162 | Transcribed locus, moderately similar to NP_113670.1 APG10 autophagy 10- |
| | | | | like (S. [symbol] |
| 39 | 1.73 | 0.03026 | CK960061 | Transcribed locus, moderately similar to NP_115602.1 zinc finger, CCHC |
| | | | | domain co [symbol] |
| 40 | 1.73 | 0.04171 | CK776888 | Transcribed locus, strongly similar to NP_081526.1 RIKEN cDNA 2010305A19 |
| | | | | gene [M [symbol] |

[symbol]

indicates data missing or illegible when filed

TABLE III
UTERINE - INCREASE

| No. | Ratio | p-value | Identifier | Gene Name |
|---|---|---|---|---|
| 1 | 185.21 | 0.00005 | BP109672 | Transcribed locus |
| 2 | 124.69 | 0.00001 | NM_173941 | myxovirus (influenza virus) resistance 2 (mouse) |
| 3 | 65.51 | 0.00017 | CB530781 | Transcribed locus |
| 4 | 57.57 | 0.00015 | NM_174366 | interferon-stimulated protein, 15 kDa |
| 5 | 48.74 | 0.00102 | CB422521 | Transcribed locus, weakly similar to NP_004326.1 bone marrow |
| | | | | stromal cell antige [symbol] |
| 6 | 48.55 | 0.00020 | CB427688 | Transcribed locus |
| 7 | 41.12 | 0.00126 | CK846889 | Transcribed locus, weakly similar to NP_004326.1 bone marrow |
| | | | | stromal cell antige [symbol] |
| 8 | 32.35 | 0.00132 | NM_174313 | fatty acid binding protein (heart) like [symbol] |
| 9 | 27.51 | 0.00005 | CB535104 | Transcribed locus |
| 10 | 21.21 | 0.00006 | CB460780 | Transcribed locus, moderately similar to XP_513514.1 similar to |
| | | | | Interferon-induc [symbol] |
| 11 | 20.81 | 0.00085 | CK955157 | Transcribed locus, moderately similar to XP_513514.1 similar to |
| | | | | Interferon-induc [symbol] |
| 12 | 20.14 | 0.00056 | CK940917 | Transcribed locus, moderately similar to XP_036729.2 ubiquitin |
| | | | | specific protease [symbol] |
| 13 | 19.90 | 0.00011 | CB433212 | Transcribed locus, moderately similar to XP_036729.2 ubiquitin |
| | | | | specific protease [symbol] |
| 14 | 19.63 | 0.00042 | CK946867 | Transcribed locus |
| 15 | 19.48 | 0.00001 | CK846137 | Transcribed locus, weakly similar to XP_515533.1 adducin 2 [Pan |
| | | | | troglodytes] |
| 16 | 19.39 | 0.00000 | CK777675 | Transcribed locus, moderately similar to XP_513514.1 similar to |
| | | | | Interferon-induc [symbol] |
| 17 | 18.89 | 0.00002 | BE756263 | Transcribed locus, strongly similar to XP_516406.1 exosome |
| | | | | component 7 [Pan trog [symbol] |
| 18 | 18.63 | 0.00011 | CB433489 | Transcribed sequence with weak similarity to protein pir: S48218 |
| | | | | (H. sapiens) S482 [symbol] |
| 19 | 18.42 | 0.00010 | CK848208 | Transcribed locus, weakly similar to XP_524747.1 similar to |
| | | | | histocompatibility 2 [symbol] |
| 20 | 17.89 | 0.00001 | BM031140 | Transcribed sequence |
| 21 | 17.65 | 0.00154 | CB533091 | Transcribed locus, moderately similar to XP_517217.1 similar to |
| | | | | Small inducible [symbol] |
| 22 | 17.30 | 0.00030 | BF440165 | Transcribed locus, moderately similar to XP_036729.2 ubiquitin |
| | | | | specific protease [symbol] |
| 23 | 15.60 | 0.00505 | NM_174816 | glycosylphosphatidylinositol specific phospholipase D1 |
| 24 | 15.40 | 0.00015 | CK960499 | 2′-5′-oligoadenylate synthetase 1 (OAS1) |
| 25 | 14.79 | 0.00154 | CK771900 | Transcribed locus, moderately similar to XP_523147.1 LOC467752 |
| | | | | [Pan troglodytes] |
| 26 | 14.65 | 0.00090 | CB432365 | Transcribed locus, moderately similar to XP_520524.1 similar to |
| | | | | DEAD/H (Asp-Glu- [symbol] |
| 27 | 14.50 | 0.00514 | CK771386 | Transcribed locus, weakly similar to NP_071430.1 28 kD interferon |
| | | | | responsive prot [symbol] |
| 28 | 14.44 | 0.00097 | CK945739 | Transcribed locus, weakly similar to XP_527300.1 similar to zinc |
| | | | | finger protein [symbol] |
| 29 | 14.12 | 0.00068 | CK966909 | Transcribed locus, moderately similar to NP_003326.2 ubiquitin- |
| | | | | activating enzyme |
| 30 | 13.32 | 0.04006 | BP110466 | Transcribed locus, strongly similar to XP_507795.1 similar to |
| | | | | Dickkopf related p [symbol] |
| 31 | 12.72 | 0.00006 | CK979617 | Transcribed locus, weakly similar to NP_004130.2 lipopolysaccharide |
| | | | | binding prot [symbol] |
| 32 | 11.61 | 0.04932 | BP107232 | Transcribed locus, strongly similar to XP_507795.1 similar to |
| | | | | Dickkopf related p [symbol] |
| 33 | 11.47 | 0.00006 | CK950711 | Transcribed locus, moderately similar to NP_057703.1 placenta- |
| | | | | specific 8 [Homo s [symbol] |
| 34 | 10.83 | 0.00025 | CB438214 | Transcribed locus, moderately similar to NP_150280.1 epithelial |
| | | | | stromal interact [symbol] |
| 35 | 10.73 | 0.00049 | NM_174007 | chemokine (C-C motif) ligand 8 |
| 36 | 10.69 | 0.00088 | NM_173940 | myxovirus (influenza) resistance 1, (murine homolog) |
| 37 | 10.66 | 0.00234 | CB419326 | Transcribed locus |
| 38 | 10.54 | 0.00094 | AW356061 | Transcribed locus, weakly similar to NP_004130.2 lipopolysaccharide |
| | | | | binding prot [symbol] |
| 39 | 10.41 | 0.01112 | BM436029 | Transcribed locus, moderately similar to NP_079537.1 lymphocyte |
| | | | | antigen 6 comple [symbol] |
| 40 | 10.18 | 0.00333 | CK846935 | Transcribed locus, moderately similar to NP_077024.1 likely ortholog |
| | | | | of mouse D1 [symbol] |
| 41 | 10.04 | 0.00122 | CK771260 | Transcribed locus, weakly similar to XP_221883.2 similar to |
| | | | | Interferon-induced g [symbol] |
| 42 | 9.41 | 0.00214 | CK940246 | Transcribed locus, moderately similar to NP_071451.2 interferon |
| | | | | induced with hel [symbol] |
| 43 | 9.35 | 0.00085 | CK770588 | Transcribed locus, weakly similar to NP_059993.2 XIAP associated |
| | | | | factor-1 [Homo [symbol] |
| 44 | 9.18 | 0.00045 | BM251565 | Transcribed locus, moderately similar to XP_290768.4 chromosome |
| | | | | 17 open reading [symbol] |
| 45 | 9.04 | 0.00018 | BE723335 | Transcribed locus, moderately similar to NP_001563.2 interferon |
| | | | | regulatory facto [symbol] |
| 46 | 8.98 | 0.00035 | CB463710 | Transcribed sequences |
| 47 | 8.95 | 0.00068 | CK776938 | Transcribed locus, moderately similar to NP_004109.1 forkhead-like |
| | | | | 18 (Drosophil [symbol] |
| 48 | 8.68 | 0.00100 | CB419688 | Transcribed locus |
| 49 | 8.46 | 0.00236 | CB450623 | Transcribed locus, strongly similar to XP_514712.1 LOC458321 [Pan |
| | | | | troglodytes] |

[symbol]

indicates data missing or illegible when filed

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for detecting a bovine pregnancy marker molecule in a bovine test animal, said method comprising:
a) obtaining a first biological sample from a bovine animal and a second control sample from a non-pregnant bovine;
b) contacting said samples with an agent having affinity for said bovine pregnancy marker molecule and
c) determining from b) the amount of said bovine pregnancy marker molecule in said first sample relative to said second sample, wherein an alteration of levels of said bovine pregnancy marker relative to those obtained from said non-pregnant bovine, is indicative of pregnancy in said test animal.

2. The method as claimed in claim 1, wherein said bovine pregnancy marker molecule is selected from the group consisting of a polypeptide, a nucleic acid and fragments thereof.

3. The method of claim 1, wherein said bovine pregnancy marker molecule is a polypeptide encoded by a nucleic acid provided in Tables I-III and said agent having affinity for said marker is an antibody.

4. The method of claim 1, wherein said bovine pregnancy marker molecule is a nucleic acid provided in Tables I-III and said agent having affinity for said marker molecule is a nucleic acid which is complementary to at least one nucleic acid provided in Tables I-III.

5. The method of claim 4, wherein said nucleic acid is selected from the group consisting of GenBank Accession number NM_{—}174313, CK960499, NM_{—}174366.1, CK947713, CB422521, CB450531, CK774949, CK848475, D87918.1, NM_{—}17401, BM258007 or a fragment thereof and said biological sample is a blood sample.

6. The method as claimed in claim 5, wherein said Bovine pregnancy inducible marker molecule is provided in SEQ ID NO: 1 or SEQ ID NO: 2.

7. The method as claimed in claim 1, wherein said biological sample is selected from the group consisting of blood, urine, uterine tissue, chorionic villi and saliva.

8. The method as claimed in claim 1, wherein said biological sample is a blood sample.

9. The method as claimed in claim 3, wherein said antibody comprises a detectable label selected from the group consisting of fluorescin, rhodamine, phycoerythrin, biotin, and strepavidin.

10. The method as claimed in claim 9, wherein said antibody is detected by a method selected from the group consisting of flow cytometric analysis, immunochemical detection and immunoblot analysis.

11. The method as claimed in claim 1, wherein said marker molecules are nucleic acids which are extracted from said biological samples and said agent having affinity for said maker comprises oligonucleotide primers which specifically hybridize to a marker encoding nucleic acid if present; wherein said method further comprises subjecting said extracted nucleic acid and primers to conditions suitable for polymerase chain reaction amplification; and assessing the resulting reaction product for an alteration in expression levels of said nucleic acid in said first sample relative to said control sample, the presence of an alteration being indicative of bovine pregnancy.

12. The method as claimed in claim 11, wherein said reaction product is assessed by a method selected from the group consisting of gel electrophoresis, restriction digest mapping, scintillation counting and filter paper assays.

13. The method as claimed in claim 12, wherein said primers comprise a detectable label.

14. The method as claimed in claim 13, wherein said detectable label is selected from the group consisting of chemiluminescent, enzymatic, radioactive, fluorescent, biotin, and stretavidin.

15. The method as claimed in claim 11, wherein said biological sample is comprises peripheral blood.

16. The method of claim 1, which is performed between days 13 to 21 of a suspected pregnancy.

17. The method of claim 16, which is performed on day 18 of a suspected pregnancy.

18. A plurality of isolated double-stranded nucleic acid molecules which are differentially expressed in bovine pregnancy shown in Tables I-III, said molecules being affixed to a solid support.

19. A plurality of isolated polypeptides encoded by the nucleic acids of claim 18.

20. An antibody immunologically specific for at least one polypeptide of claim 19, affixed to a solid support.

21. A kit for detecting bovine pregnancy in a biological sample, said kit comprising at least one first reagent for detecting a bovine pregnancy marker molecule.

22. The kit of claim 21, wherein said bovine pregnancy marker molecule is selected from the group consisting of a polypeptide, a nucleic acid molecule, and fragments thereof.

23. The kit of claim 21, wherein said bovine pregnancy marker molecule is a polypeptide and wherein said first reagent is an antibody or fragment thereof having affinity for said bovine pregnancy marker molecule.

24. The kit of claim 23, wherein said antibody is detectably labeled.

25. The kit of claim 23, wherein the kit further comprises at least one second reagent for detecting a bovine pregnancy marker molecule-antibody immunocomplex, if present, in said biological sample.

26. The kit of claim 23, wherein said antibody or fragment thereof is in solution.

27. The kit of claim 24, wherein said detectable label is selected from the group consisting of fluorescein, rhodamine, phycoerythrin, biotin, and strepavidin.

28. The kit of claim 25, wherein said second reagent is selected from the group consisting of flow cytometric reagents, immunochemical detection reagents, and immunoblot reagents.

29. The kit of claim 21, wherein said bovine pregnancy marker molecule is a nucleic acid molecule or a fragment thereof.

30. The kit of claim 29, wherein said bovine pregnancy marker molecule is a nucleic acid molecule and wherein said first reagent is nucleic acid molecule which is complementary to said bovine pregnancy marker molecule.

31. The kit of claim 30, wherein said first reagent is a primer.

32. The kit of claim 31, further comprising:
a) a polymerase enzyme suitable for use in polymerase chain reaction;
b) buffers and nucleotides suitable for performing amplification reactions;
c) a DNA sample comprising a positive control; and
d) optionally, an instruction protocol.

33. The kit of claim 30, wherein said primers are complementary to SEQ ID NO: 1 or SEQ ID NO: 2.

34. The kit of claim 30, wherein said primer comprises a detectable label.

35. The kit of claim 34, wherein said detectable label is selected from the group consisting of: chemilluminescent, enzymatic, radioactive, fluorescent, biotin, and streptavidin.

36. The kit of claim 30, further comprising at least one second reagent selected from the group consisting of gel electrophoresis reagents, restriction digest mapping reagents, scintillation counting reagents, and filter paper assays reagents.

37. The kit of claim 32, further comprising:
e) an antibody or fragment thereof, optionally detectably labeled, immunologically specific for a region of a polypeptide encoded by SEQ ID NO: 1 or SEQ ID NO: 2; and
f) at least one reagent for detecting a bovine pregnancy marker molecule-antibody immunocomplex, if present, in said biological sample.
